SEQUENCE LISTING (5 sequences)

SEQ ID NO: 1 - protein, 16 aa, synthetic construct
MASGVAVSDGVIKVFN

SEQ ID NO: 2 - protein, 16 aa, synthetic construct
NFVKIVGDSVAVGSAM

SEQ ID NO: 3 - protein, 16 aa, synthetic construct
RQIKIWFQNRRMKWKK

SEQ ID NO: 4 - protein, 32 aa, synthetic construct
MASGVAVSDGVIKVFNRQIKIWFQNRRMKWKK

SEQ ID NO: 5 - protein, 32 aa, synthetic construct
NFVKIVGDSVAVGSAMRQIKIWFQNRRMKWKK

(19) United States
(12) Patent Application Publication
(10) Pub. No.: US 2026/0108640 A1
(43) Pub. Date: Apr. 23, 2026

(54) Peptide Composition for Cancer Approach and Diagnosis

(71) Applicant: National Yang Ming Chiao Tung University, Hsinchu (TW)

(72) Inventors: Yi-Jang LEE, Hsinchu (TW); Min-Ying LIN, Hsinchu (TW); Chun-Yi WU, Hsinchu (TW); Jyh-Der LEU, Hsinchu (TW)

(21) Appl. No.: 18/923,581

(22) Filed: Oct. 22, 2024

(51) Int. Cl. A61K 51/08; A61K 45/06; A61P 35/00

(52) U.S. Cl. CPC A61K 51/08; A61K 45/06; A61P 35/00; A61K 2121/00; A61K 2123/00

(57) ABSTRACT

A peptide sequence mimicking endogenous cofilin-1 incorporates transmembrane amino acid sequences to enhance cellular uptake of the peptide drug. When cofilin peptide drugs are introduced into cancer cells, they affect cancer cell migration and invasion abilities. Furthermore, the peptide sequence can be conjugated with a chelator for cancer diagnosis.

### REFERENCE TO AN ELECTRONIC SEQUENCE LISTING

The content of the electronic sequence listing (sequence listing.xml; Size: 5,230 bytes; and Date of Creation: Sep. 19, 2024) is herein incorporated by reference in its entirety.

### BACKGROUND OF THE INVENTION

### Technical Field of the Invention

The present invention relates to a peptide and its use in cancer treatment. In particular, the present invention discloses a cofilin peptide, alone or in combination with a chelator, for use in inhibiting the transfer of cancer cells and for diagnosing cancer cells.

### Background

Lung cancer is the most common malignancy and the leading cause of cancer mortality in the world. Approximately 75% of lung cancers are non-small-cell lung cancer (NSCLC), with adenocarcinoma being one of the main types of NSCLC (Molina J R et. al., Mayo Clin Proc 2008:83:584-94). The determination of stage in NSCLC has important therapeutic and prognostic implications. In general, distant metastasis remains the major cause of failure for NSCLC after treatment. As the patients are diagnosed with distant metastasis, they may be treated the first-line therapy with small molecules targeting specific tyrosine kinase receptors and immune checkpoint inhibitors (Yang C Y et. al., Annu Rev Med 2020:71:117-36). However, more than 50% of lung cancer patients are diagnosed at an advanced stage, which is strongly associated with poor prognosis (Eberhardt W E et. al., J Thorac Oncol 2015:10:1515-22). The 5-year survival rate for metastatic NSCLC is approximately 7% (Bray F et. al., CA Cancer J Clin 2018:68:394-424). Therefore, it is important to develop a specific strategy of anti-invasion and antimetastatic drugs for the treatment of cancer.

Compared to existing monoclonal antibody targeted therapies, peptide drugs offer several advantages, such as lower development costs and minimal noticeable side effects. These benefits enhance treatment options and reduce the financial burden on patients. The advantages of using peptides as pharmaceutical compounds include mimicking natural pathways, antigenicity, high specificity, and a high metabolic rate. There are many post-transcriptional modifications of molecules that lead to carcinogenesis in intracellular expressed, so it should be important to consider peptide reagents that target cellular proteins for cancer therapeutic purposes (Wang L et. al., Signal Transduct Target Ther 2022; 7:48).

Destabilization of actin dynamics is a major strategy to impede the rapid proliferation of tumor cells. The actin depolymerizing factor (ADF)/cofilin protein family is important to actin dynamics and is known as intracellular actin-modulating protein (Bamburg J R et. al., Annu Rev Cell Dev Biol 1999; 15:185-230; Maciver S K et. al., Genome Biol 2002; 3:reviews3007). Cofilin-1 (CFL-1) is the most abundant isoform of the ADF/cofilin family, predominantly expressed in non-muscle tissues. It plays a vital role in cellular dynamics by accelerating the turnover of actin filaments through depolymerizing or severing them, thereby influencing various cellular functions, including cell motility, morphogenesis, and cytokinesis (Kanellos G et. al., J Cell Sci 2016; 129:3211-8; Tsai C H et. al., Aging Cell 2021; 20:e13288; Advedissian T et. al., Nat Commun 2024; 15:1949). Overexpression of cofilin-1 has been found in several advanced cancerous tissues, including lung cancers, colorectal cancer, prostate cancer, and bladder cancer (Peng X C et. al., PLOS One 2011; 6:e27309; Mousavi S et. al., J Gastrointest Oncol 2018; 9:791-6; Lu L I et. al., Oncol Lett 2015; 9:2757-61; Wang F et. al., Oncotarget 2017; 8:92043-54). However, the role of cofilin-1 phosphorylation in regulating cancer metastasis remains to be fully elucidated.

### SUMMARY OF INVENTION

Therefore, the present invention provides a peptide and a therapeutic method for cancer treatment.

In one aspect, the present invention provides a pharmaceutical composition comprising a peptide with an amino acid sequence consisting of SEQID NO: 1.

In some embodiments, the pharmaceutical composition comprises a cell-membrane penetrating peptide.

In some embodiments, the cell-membrane penetrating peptide selected from any one of:
(i) an antennapedia class peptide;
(ii) a protegrin class peptide;
(iii) a HIV-TAT class peptide;
(iv) an amphipathic class peptide selected from an amphipathic and net positively charged peptide and a proline-rich amphipathic peptide;
(v) a peptide exhibiting high α-helical content;
(vi) a peptide comprising oligomers of basic amino acids;
(vii) pVEC;
(viii) a calcitonin-derived peptide; and
(ix) an amphiphilic cyclic cell penetrating sequence.

In some embodiments, the cell-membrane penetrating peptide is the antennapedia class peptide.

In some preferred embodiments, the cell-membrane penetrating peptide consists of SEQID NO: 3.

In some embodiments, the peptide is conjugated with a chelating agent selected from the group consisting of DTPA, HEDP, MGDA, DTPMP and combination thereof.

In some preferred embodiments, the peptide is conjugated with diethylene triamine pentaacetic acid (DTPA).

In some embodiments, the chelating agent chelates a radionuclide.

In some embodiments, the radionuclide is selected from the group consisting of ¹⁷⁷Lu, ^{186/188}Re, ⁶⁷Cu, ^{60/64}Cu, ⁹⁰Y, ⁸⁶Y, ^{44/43/47}Sc, ^{203/212}Pb, ^{67/68}Ga, ^{155/157}Gd, ²¹²Bi, ²²⁵Ac, ²²³Ra, ^{99m}Tc, and ¹¹¹In.

In some preferred embodiments, the radionuclide is ^{99m}Tc, or ¹¹¹In.

In some embodiments, the pharmaceutical composition further comprises an anti-cancer agent selected from the group consisting of etoposide, doxorubicin, daunorubicin, epirubicin, bleomycin, rituxan, gemcitabine, nivolumab, pembrolizumab, and ipilimumab.

In another aspect, the present invention provides a method for treating a cancer disease in a subject, comprising administering to the subject the said peptide.

In another aspect, the present invention provides a method for diagnosing a cancer disease in a subject, comprising administering to the subject the said peptide: imaging the subject to detect a signal emitted by the radionuclide.

In some embodiments, the cancer disease exhibits overexpression of the LIMK gene.

In some embodiments, the cancer disease is selected from the group consisting of colon adenocarcinoma, lymphoid neoplasm diffuse large B-cell lymphoma, esophageal carcinoma, head and neck squamous cell carcinoma, kidney renal papillary cell carcinoma, lung adenocarcinoma, pancreatic adenocarcinoma, rectum adenocarcinoma, skin cutaneous melanoma, stomach adenocarcinoma, thymoma, and uterine carcinosarcoma.

### BRIEF DESCRIPTION OF THE DRAWINGS

FIG. 1A compares cell viability in Beas2B, CL1-0, CL1-5, A549 and H1299 cells following treatment with the CFL Ser3 peptide, as assessed by MTT assay.

FIG. 1B (Left) presents Western blots analyses of CFL-1, pCFL-1, E-cadherin, N-cadherin, Vimentin and GAPDH expression after treatment with different doses of specific peptide. (Right) shows the quantification of pCFL-1/CFL-1, E-cadherin, N-cadherin and vimentin with different dose of CFL Ser3 peptide treatment.

FIG. 1C compares the actin polymerization rate and depolymerization rates in CL1-5 cells with either RV (Reverse sequence of CFL peptide) cofilin peptide or CFL Ser3 peptide treatment.

FIG. 1D (Left) shows representative images from wound healing assay of CL1-5 cells treated with different doses of RV cofilin peptide or CFL Ser3 peptide. The right panel provides quantification of the migration abilities of CL1-5 cells.

FIG. 1E (Left) displays representative images from Matrigel transwell assay of CL1-5 cells treated with different doses of RV cofilin peptide or CFL Ser3 peptide. The right panel provides the quantification of the invasion abilities of CL1-5 cells.

FIG. 1F shows the effects of CFL Ser3 peptide on the expression of pCFL-1, CFL-1 and LIMK2 protein, with or without a combination of TGF-β treatment in A549 cells.

FIG. 1G (upper) shows representative images from Matrigel transwell assay of A549 cells with or without a combination of TGF-β and CFL Ser3 peptide treatment. The lower panel presents the invasion abilities of invaded cells.

FIG. 1H (upper) displays immunofluorescence images of fluorescein-conjugated phalloidin of A549 cells with or without a combination of TGF-β and CFL Ser3 peptide treatment. The lower panel shows the quantification of the fluorescence intensity for each treatment group. *: p<0.05.

FIG. 2A shows the Radio-thin layer chromatography analysis of free In-111 or Tc-99m, and CFL Ser3 peptide labeled with In-111 or Tc-99m after DTPA challenge and purification.

FIG. 2B shows In vitro stability of In-111 or Tc-99m labeling with CFL Ser3 peptide under different storage conditions.

FIG. 2C (upper) shows Western blots analyses for the expression of LIMK2, pCFL-1 and GAPDH in CL1-5, A549 and H1299 cells. The lower panel displays the cellular uptake of ¹¹¹In-DTPA-CFL Ser3 peptide at 30-, 60- and 180 mins post-incubation in CL1-5, H1299 and A549 cells.

FIG. 2D shows cellular uptake of ¹¹¹In-DTPA-RV cofilin peptide or ¹¹¹In-DTPA-CFL Ser3 peptide at different incubation time in CL1-5 cells.

FIG. 2E shows biodistribution analysis of ¹¹¹In-DTPA-CFL Ser3 peptide in CL1-5 tumor bearing mice.

FIG. 2F shows a Nano SPECT/CT imaging of CL1-5 tumor-bearing mice injected with ^{99m}Tc-DTPA-CFL Ser3 peptide at 3 hours post-injection (MIP=maximum intensity projection; Sag.=Sagittal plane; Cor.=Coronal plane; Tra.=Transverse plane). *: p<0.05.

FIG. 3A exhibits the experimental scheme for the orthotopic lung cancer model, which was treated with intravenous injection of CFL Ser3 peptide.

FIG. 3B (Left) shows bioluminescence imaging of lung cancer responses to CFL Ser3 peptide treatment compared to solvent control after 20 days treatment. The right panel presents the quantification of photon signaling from CFL Ser3 peptide treatment versus solvent control, measured using the In Vivo Imaging System (IVIS).

FIG. 3C (Left) shows a representative bioluminescence images and microscopic images evaluated by the IVIS system. The right panel presents the quantification of ex-vivo bioluminescence signal after CFL Ser3 peptide treatment compared to the solvent control, as measured by IVIS.

FIG. 3D shows a representative microscopic image of H&E staining and the number of metastatic tumors counted.

FIG. 3E shows a representative microscopic image of IHC staining of lung tumor tissue after CFL Ser3 peptide treatment.

FIG. 3F shows a representative microscopic image of H&E staining of normal tissue organ after CFL Ser3 peptide treatment.

FIG. 3G shows the body weight of the mice in CFL Ser3 peptide treatment group and solvent control group during experimental course.

FIG. 3H exhibits a Kaplan-Meier plot showing survival times of mice treated with solvent control (saline) versus CFL Ser3 peptide. *: p<0.05.

FIG. 4. exhibits a schematic model illustrating the dual role of CFL Ser3 peptide in lung cancer.

FIG. 5A shows the effects of CFL peptide treatment on the expression of γ-H2AX, ATM activation and CFL phosphorylation with or without a combination of radiation.

FIG. 5B shows the detection of γ-H2AX foci formation in the nucleus with or without a combination of TGF-β and CFL peptide treatment.

FIG. 5C shows Fluorescence-based DNA repair analysis for non-homologous end joining (NHEJ) after 20 μM of CFL Ser3 peptide with 8 Gy irradiation

FIG. 5D shows the quantification of GFP ratio normalized against DsRed.

FIG. 5E compares cell survival fractions between irradiated H1299 cells treated with solvent control, RV peptide, or CFL Ser3 peptide, as determined by the colony formation assay.

### DETAILED DESCRIPTION OF THE INVENTION

Unless defined otherwise, all technical and scientific terms used herein have the same meaning as commonly understood by a person skilled in the art to which this invention belongs.

As used herein, the singular forms “a”, “an”, and “the” include plural referents unless the context clearly dictates otherwise. Thus, for example, reference to “a sample” includes a plurality of such samples and equivalents thereof known to those skilled in the art.

The term “peptide” is used herein to designate a series of amino acid residues, connected one to the other typically by peptide bonds between the alpha-amino and carbonyl groups of the adjacent amino acids.

Furthermore, the term “peptide” shall include salts of a series of amino acid residues, connected one to the other typically by peptide bonds between the alpha-amino and carbonyl groups of the adjacent amino acids. Preferably, the salts are pharmaceutical acceptable salts of the peptides, such as, for example, the chloride or acetate (trifluoroacetate) salts.

The term “peptide” shall also include “oligopeptide”. The term “oligopeptide” is used herein to designate a series of amino acid residues, connected one to the other typically by peptide bonds between the alpha-amino and carbonyl groups of the adjacent amino acids.

Pharmaceutical compositions for use in accordance with the present invention may be formulated in a conventional manner using one or more physiologically acceptable carriers comprising excipients and auxiliaries which facilitate processing of the active compounds into preparations which can be used pharmaceutically. Proper formulation is dependent upon the route of administration chosen.

### Materials and Methods

The practice of the present invention will employ technologies comprising conventional techniques of cell biology and cell culture, which are within the ordinary skills of the art. Such techniques are explained fully in the literature.

### Cell Cultures

The human bronchial epithelium cells line Beas2B (American Type Culture Collation, Manassas, VA, USA) were grown in keratinocyte-SFM medium (Life Technologies Inc., Carlsbad, CA, USA). The cell lines H1299, H441, H292, H928, H1355, CL1-0 and CL1-5 were grown in RPMI1640 medium (Life Technologies Inc., Carlsbad, CA, USA) supplemented with 10% FBS. CL1-0 was a sibling cell line of CL1-5 with different metastatic characteristics. The human lung adenocarcinoma cell line A549 was grown in DMEM (Life Technologies Inc., Carlsbad, CA, USA) supplemented with 10% FBS. All cells were incubated under a humidified atmosphere of 5% CO2 at 37° C.

Lung cancer cells were used to assess the treatment effects of the Cofilin Ser3 peptide, but this is not limited to these lesions. Typically, cancers with overexpression of LIMK include Colon adenocarcinoma, Lymphoid Neoplasm Diffuse Large B-cell Lymphoma, Esophageal carcinoma, Head and Neck squamous cell carcinoma, Kidney renal papillary cell carcinoma, Lung adenocarcinoma, Pancreatic adenocarcinoma, Rectum adenocarcinoma, Skin Cutaneous Melanoma, Stomach adenocarcinoma, Thymoma, and Uterine Carcinosarcoma can also be used as cancer cell model for assess the treatment effect of Cofilin Ser3 peptide.

### Cofilin Mimetic Peptide Design and Treatment

The cofilin Ser3 peptide and cofilin rev-Ser3 peptide were synthesized by BioTools Co., Ltd (Taiwan) using solid phase peptide synthesis. The peptides contained 16 amino acids by mimicking the N-terminal sequence of human cofilin (MASGVAVSDGVIKVFN) and reverse sequence of cofilin (NFVKIVGDSVAVGSAM), together with cell penetrating sequence (RQIKIWFQNRRMKWKK). All peptides were analyzed by high-performance liquid chromatography (HPLC) on Sinochrom ODS-BP column. Solvents A and B were 0.1% trifluoroacetic (TFA) water and 0.1% TFA acetonitrile, respectively. Linear gradients of Solvent B to A at 1.0 ml/min flow rate were used for elution, with UV detection at 200 nm. Purities of the purified peptides were over 95% and characterized by mass spectrometry. Each synthetic peptide was dissolved in sterile double-distilled water to a concentration of 2.5 mM and added to the cultures to the indicated final concentrations.

### Cell Viability Measurements

Cells were seeded in 96-well plates after treated with different concentration of peptide for 24 hours. After removal of the supernatant, 5 mg/ml 3-(4,5-dimethylthiazol-2-yl)-2,5-diphenyltetrazolium bromide (MTT) solution (Sigma-Aldrich, St. Louis, MO, USA) was mixed with serum-free medium and incubated for 2˜4 hours. The produced crystals were dissolved in dimethyl sulfoxide (DMSO). The plate was measured at 570 nm using an ELISA reader (Bio-Tek Instrument, USA).

Radiolabeling of ¹¹¹In-DTPA-cofilin peptide and ^{99m}Tc-DTPA-cofilin peptide

A radionuclide chelator was conjugated to the targeting peptide to form a peptide-based radiopharmaceutical. The modification of DTPA was made at the N-terminal by solid-phase synthesis (Synbio tech., NJ, USA). ¹¹¹In-InCl3 (purchased from the Institute of Nuclear Energy Research) and DTPA-cofilin peptide (¹¹¹In-DTPA-cofilin peptide=10 μCi/1 μg) were added to a vial containing citrate buffer (0.1 M, pH=5) and the mixture was allowed to react at 40° C. for 30 min. The mixture was centrifuged at 12,000 g for 10 min; For radiochemical synthesis of ^{99m}Tc-DTPA-cofilin peptide, 20 μl of DTPA-cofilin peptide (0.1 mg/ml) and 25 μl of SnCl₂ (1 mg/ml in 0.01M HCl) were added and mixed. After 15 min incubation, 2 mCi Tc-99m pertechnetate (Na+^{99m}TcO4-, provided from Taipei Veterans General Hospital) was added into vial and incubated for 20 min at room temperature. The mixture was centrifuged at 12,000 g for 10 min. The labeling efficiency and radiochemical purity were determined by radio-thin layer chromatography (radio TLC) using sodium citrate buffer (0.5 M, pH=5.0) as the mobile phase. To evaluate the stability, the radiolabeled peptides were incubated in saline and FBS at room temperature and 37° C. after radiolabeling. The percentages of intact ¹¹¹In-DTPA-cofilin peptide or ^{99m}Tc-DTPA-cofilin peptide were determined by radioTLC at each time point.

### Cellular Uptake Assay Studies

Cells were seeded into a 6-well plate at a density of 1×10⁶ cells/well and incubated overnight. The culture medium was replaced by fresh medium containing ¹¹¹In-DTPA-cofilin mimetic peptide (1 μCi/ml). At different time points post incubation, the medium and washing PBS were collected in a counting tube. Furthermore, cells were trypsinized and collected by washing with PBS into another counting tube. The cellular uptake of ¹¹¹In-DTPA-cofilin mimetic peptide was expressed as the percentage of administrated dose per one million cells (% AD/10⁶ cells).

### Animal Studies

Age-matched severe combined immunodeficient NOD/SCID (NOD.CB17-Prkdcscid/JNarl) male mice at 6-8 weeks old were obtained from National Laboratory Animal Center, Taiwan, and used in the present invention. All mice were housed in individually ventilated cages (IVC) facility (with controlled temperature at 24° C.; controlled humidity at 50%, and maintained on a daily 12 h light, 12 h dark cycle. The animals were also provided with free access to food and water, and the bedding was replaced twice a week. Animal health and behavior were daily monitored. To evaluate lung colony-forming ability, 1×10⁶ cells CL1-5 cells with luciferase gene were resuspended in 100 μL of PBS and injected into the lateral tail vein. Imaging of tumor growth and development under different experimental conditions were investigated using In vivo Imaging System Lumina X5 (IVIS Lumina X5, PerkinElmer, Inc., Hopkinton, USA). In brief, mice were intraperitoneal injected with 150 mg/kg D-luciferin (Caliper Co., Hopkinton, MA, USA), and anesthetized using 2-4% isoflurane in IVIS system for imaging acquisition. Lung nodule formation was quantified after H&E staining using Axioscan 7 Slide Scanner (Carl Zeiss Microscopy GmbH, Jena, Germany) at the endpoint. For lung orthotopic xenograft model, 1×10⁶ CL1-5 cells with luciferase gene in 10 μL PBS were resuspended and mixed with 10 μL Matrigel and intrapulmonary injection transplanted into the right lung of the mice. After tumor inoculation, body weights were measured twice a week. Treatment was stopped when the bodyweight of the animals decreased to below 80% of their starting body weight. The mice were euthanized by CO₂ filled in the air chamber. All animal experiments were conducted in accordance with Guide for the Use and Care of Laboratory Animals (Animal Research: Reporting of In Vivo Experiments guidelines), and the animal protocol (IACUC No. 1110601) was approved by the Experimental Animal Committee, National Yang Ming Chiao Tung University, Taiwan.

Animal SPECT/CT Imaging of ^{99m}Tc-DTPA-Cofilin Peptide

After injection of 1 mCi/100 μl ^{99m}Tc-DTPA-Cofilin peptide via intravenous of mice, static images were performed with the nanoSPECT/CT imaging modality (Mediso, Hungary). SPECT/CT images were acquired at 3 h after injection in the lung cancer mice model. Standard uptake values (SUV) of tumor were calculated using AMIDE software (Version 1.0.5). The animal SPECT/CT images were acquired at Chang Gung Memorial Hospital and National Atomic Research Institute, Taoyuan City, Taiwan.

### EXAMPLES

The other characteristics and advantages of the present invention will be further illustrated and described in the following examples. The examples described herein are using for illustrations, not for limitations of the invention.

### Embodiment 1: The Cofilin Ser3 Peptide Inhibits Lung Cancer Cell Metastatic Ability by Regulating Endogenous CFL-1 Phosphorylation

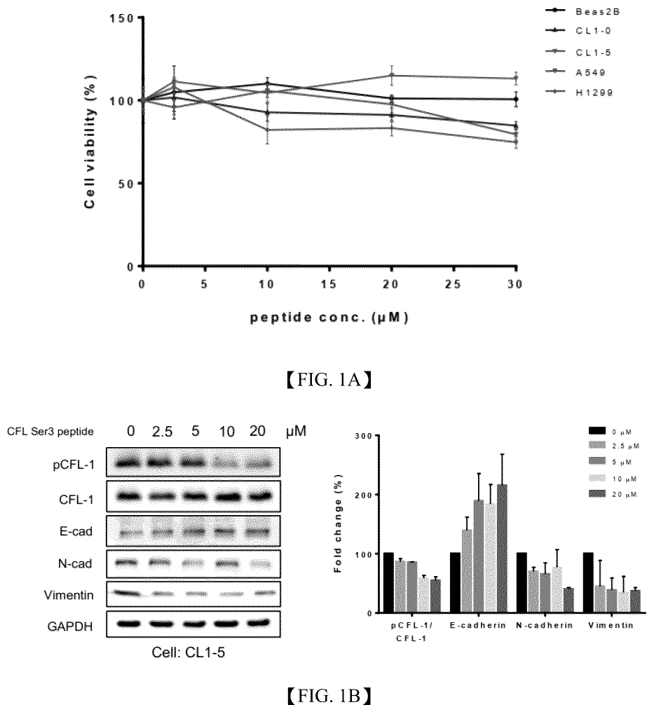

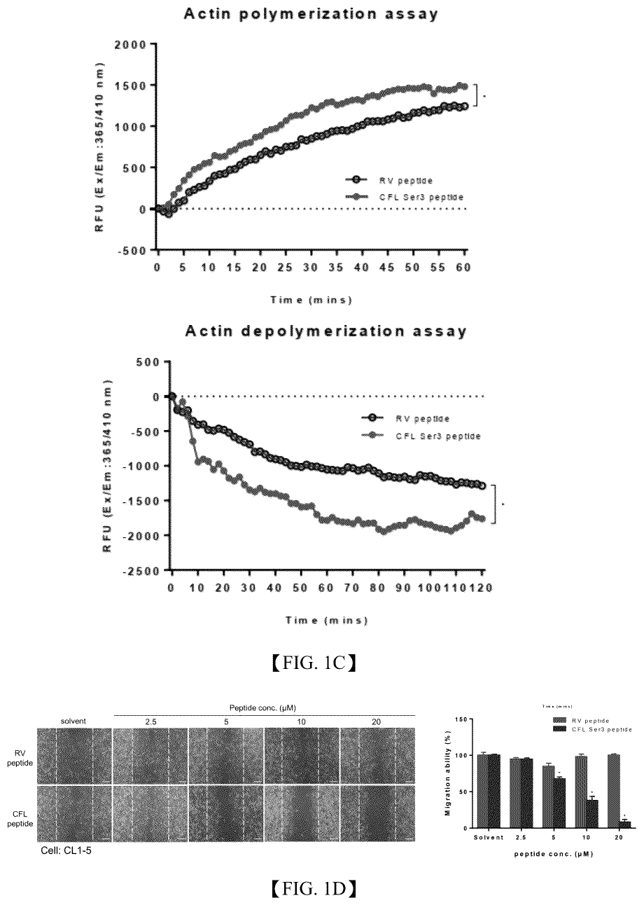

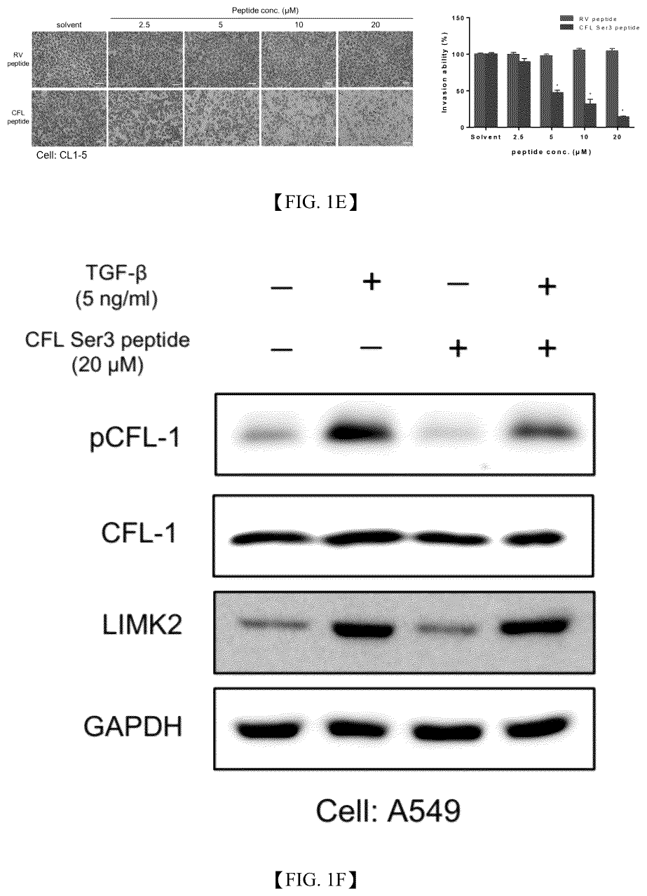

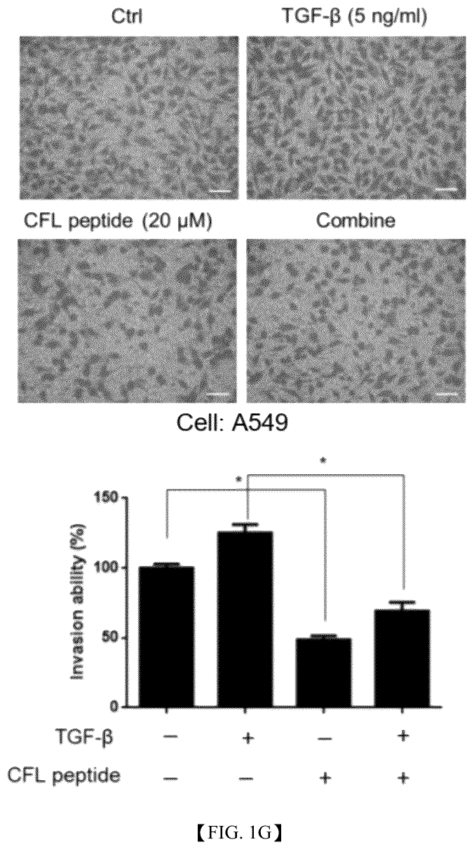

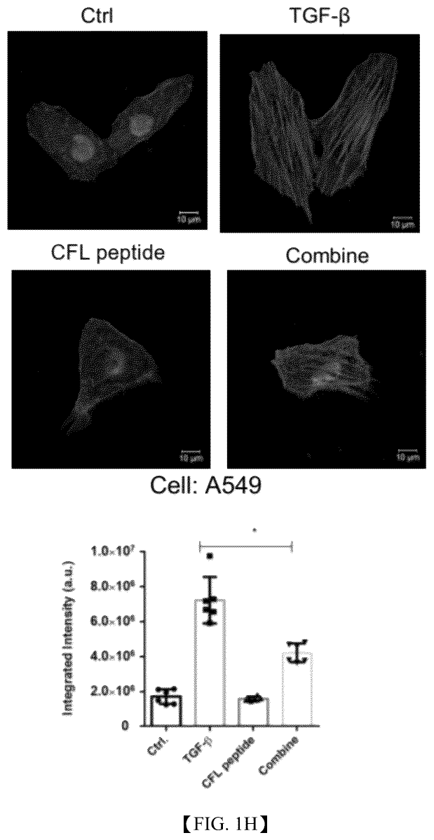

To interfere the phosphorylation of cofilin-1 in lung cancer cells, the peptidomimetic approach was used to modulate endogenous cofilin-1 activity. The peptide contained with the first 16 amino acids mimicking the cofilin protein sequence, or its reverse sequence, along with a penetrating sequence for cell internalization. As shown in FIG. 1A, the peptide did not induce cytotoxicity in dose-dependent experimental system using normal bronchial cells and various lung cancer cell lines. To confirm that the cofilin mimetic peptide can destabilize the actin dynamics, the peptide was utilized to validate whether the suppression of endogenous CFL-1 phosphorylation can influence lung cancer cell motility. As shown in FIG. 1B, CFL Ser3 peptide treatment was able to reduce the endogenous levels of pCFL-1 and reversed the levels of EMT-related markers in lung cancer cells. Next, the rates of actin polymerization and depolymerization were compared between lung cancer cells treated with or without CFL Ser3 peptide using pyrene-conjugated actin polymerization assay. The results showed that the cell lysates treated with CFL Ser3 peptide demonstrated higher actin polymerization rate compared to the lysates treated with RV cofilin peptide. On the other hand, the actin depolymerization rate was significantly decreased with CFL Ser3 peptide treatment (FIG. 1C). The CFL Ser3 peptide was also able to suppress the migration and invasion ability in a dose-dependent manner of NSCLC cells, compared to the reverse peptide treatment (FIGS. 1D and 1E).

Additionally, TGF-β-induced EMT was triggered in lung cancer cells and this process was combined with CFL Ser3 peptide treatment. The western blot analysis demonstrated that CFL Ser3 peptide could inhibit TGF-β-induced CFL-1 phosphorylation in lung cancer cells (FIG. 1F). As shown in FIGS. 1G and 1H, cofilin peptide could not only inhibit cell invasion ability, but also reduce actin filament and stress fiber formation by targeting CFL Ser3 phosphorylation, as assessed by phalloidin staining.

### Embodiment 2: The CFL Ser3 Peptide can be Phosphorylated by LIMK2 and Accumulates in Lung Cancer Cells

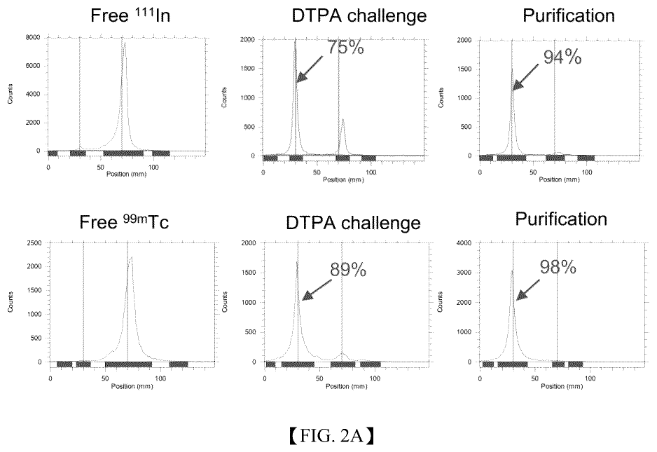

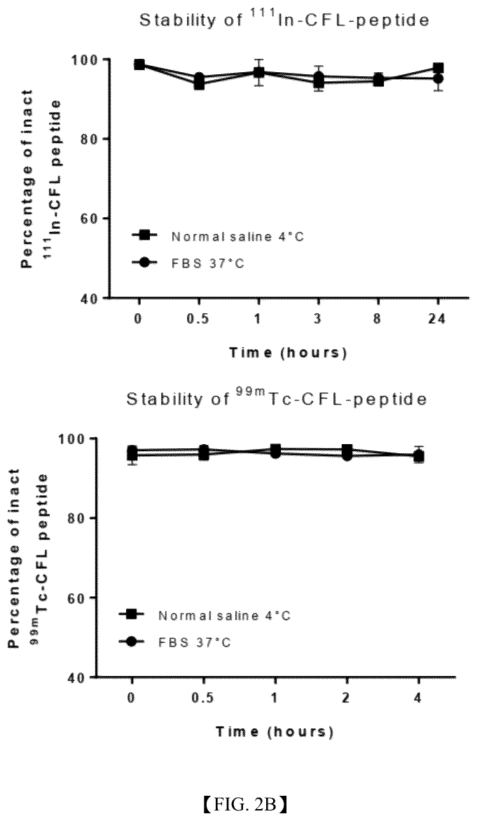

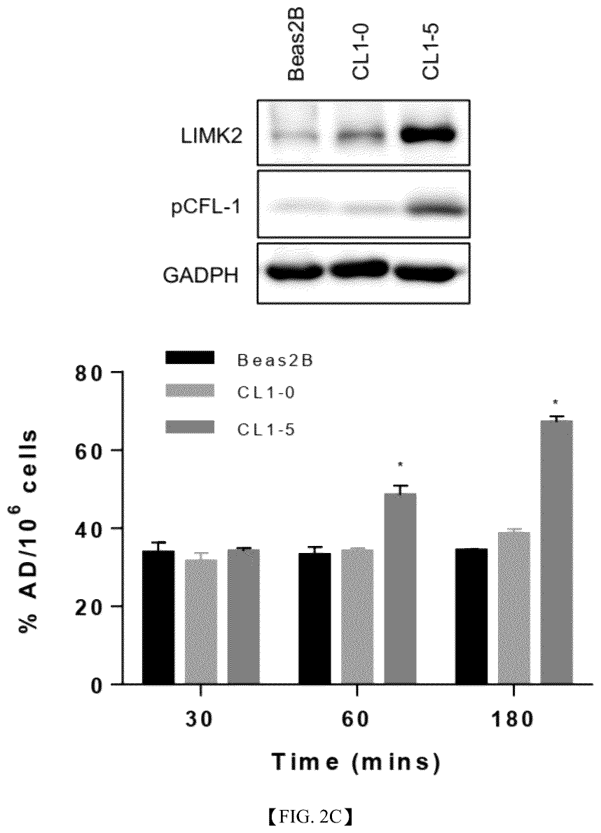

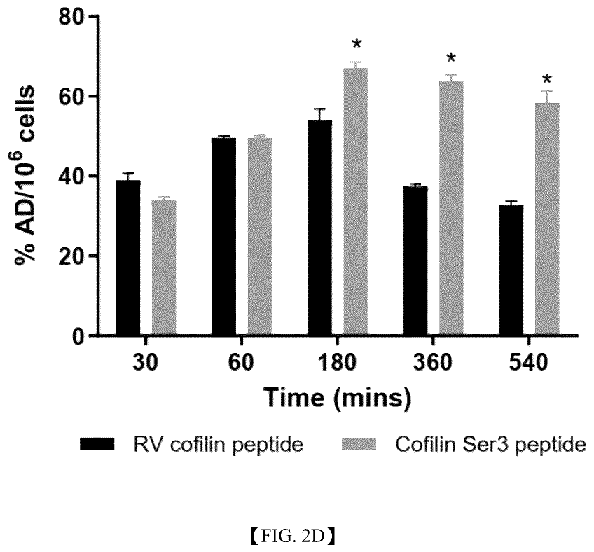

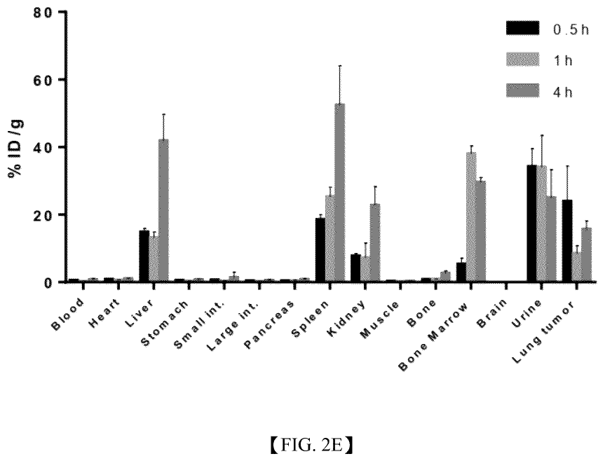

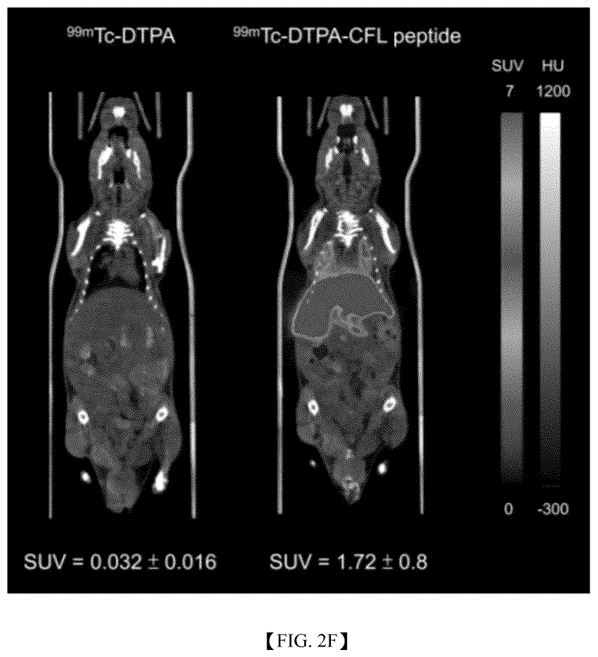

To evaluate the potential of CFL mimetic peptides for targeting tumors, the peptide was radiolabeled to trace its location both in vitro and in vivo. The cofilin mimetic peptide, conjugated with DTPA, was created using solid phase peptide synthesis and labeled with ¹¹¹In or ^{99m}Tc. After excess DTPA was removed and purification was performed by centrifugation, the radiochemical purity of ¹¹¹In-cofilin Ser3 peptide and ^{99m}Tc-cofilin Ser3 peptide was 95.88±1.93% and 95.76±2.63%, respectively (FIG. 2A). In the stability tests, the percentage of intact ¹¹¹In-CFL Ser3 peptide remained above 90% after 24 h of incubation in either normal saline or FBS (FIG. 2B). Western blot analysis revealed varying levels of endogenous LIMK2 and phosphorylated CFL-1 across three lung cancer cell lines. As shown in FIG. 2C, H1299 and CL1-5 cells increased the uptake of the CFL Ser3 peptide within 180 minutes, whereas A549 cells did not. The cellular uptake of ¹¹¹In-CFL Ser3 peptide and ¹¹¹In-RV cofilin peptide increased with time, reaching a maximum accumulation at 180 minutes post incubation.

Notably, the ¹¹¹In-CFL Ser3 peptide maintained a high level of accumulation throughout the 540 minutes study period in CL1-5 cells (FIG. 2D). The distribution of the ¹¹¹In-CFL Ser3 peptide after intravenous injection in CL1-5 tumor-bearing mice is shown as the percentage of the injected dose per gram tissue (% ID/g) (FIG. 2E). SPECT/CT imaging of ^{99m}Tc-CFL Ser3 peptide was obtained 3 h after intravenous injection. The ^{99m}Tc-CFL Ser3 peptide accumulated in CL1-5 tumor-bearing mice, expressed 1.726±0.8 as SUVs (FIG. 2F). These results suggest that the cofilin Ser3 peptide is phosphorylated by LIMK2 within lung cancer cells, leading to its accumulation within the cells as it cannot exit to the cell membrane.

### Embodiment 3: Cofilin Ser3 Peptide Inhibits Lung Cancer Metastasis In Vivo

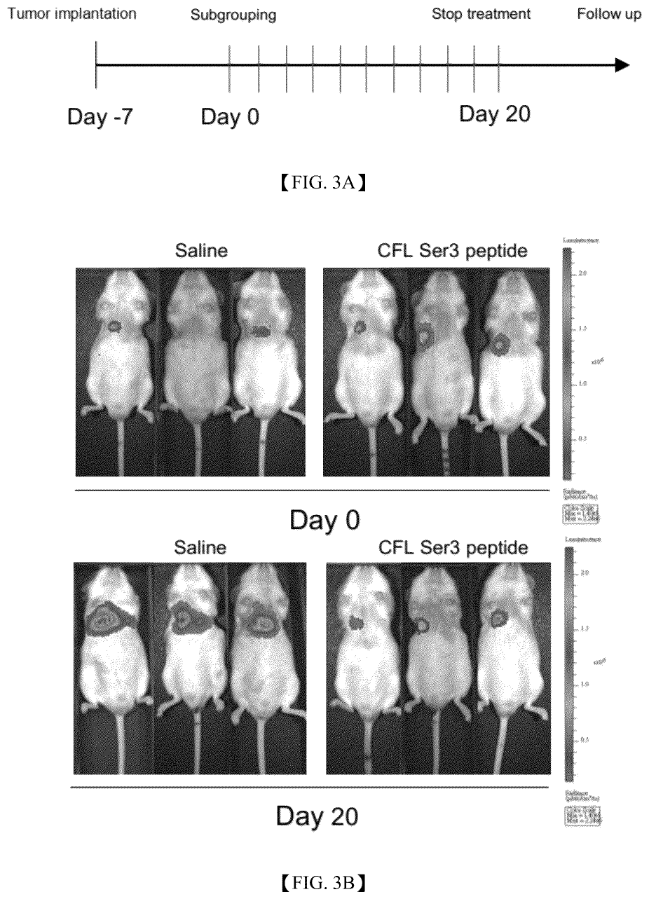

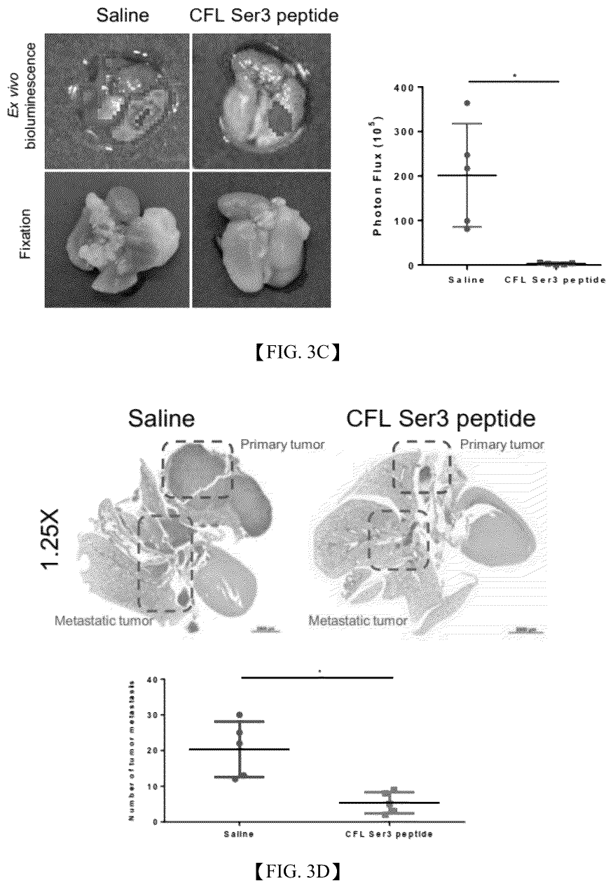

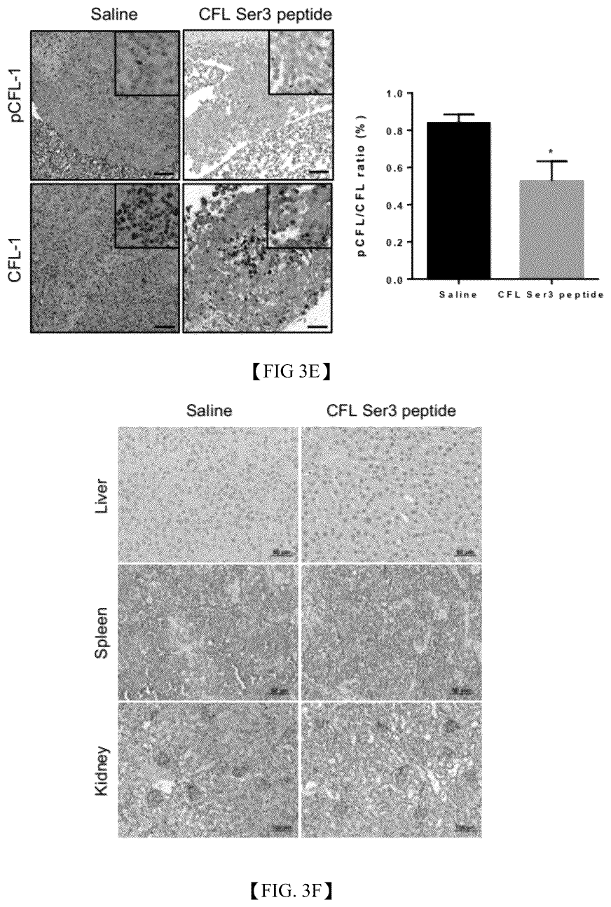

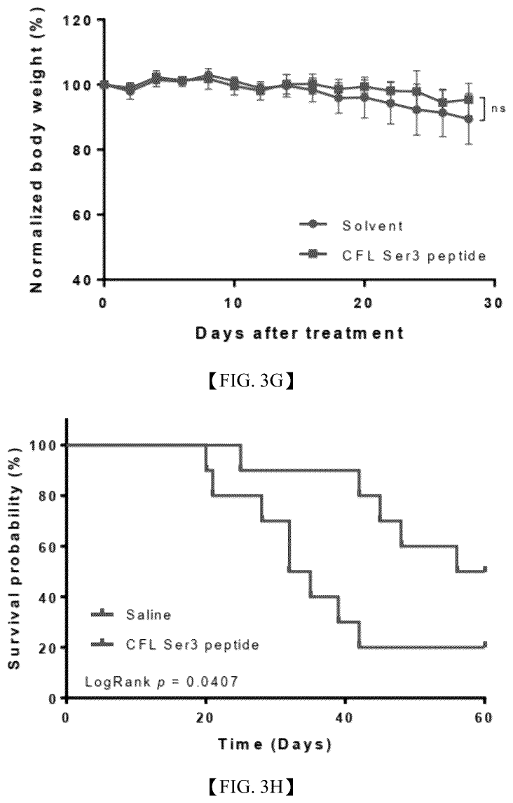

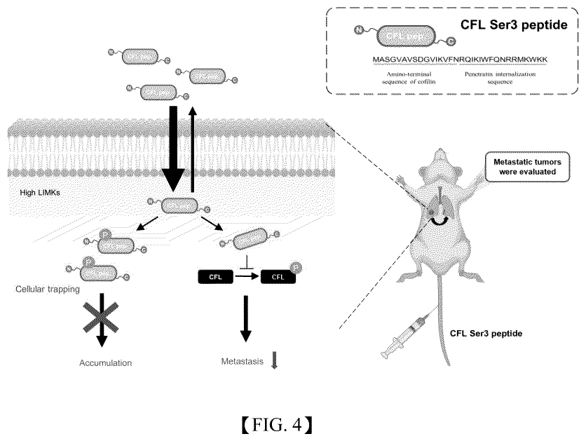

To examine the therapeutic efficacy of cofilin peptide treatment in lung cancer metastasis, an orthotopic xenograft lung cancer model in CL1-5 with luciferase gene was established. The cells were administered via intrapulmonary injection into right lungs of NOD-SCID mice and treated with 10 mmol/L/kg of cofilin Ser3 peptide once every two days via intravenous injection (FIG. 3A). The IVIS spectrum showed that the cofilin Ser3 peptide could inhibit the lung cancer invasion from right lobes to the left lobes of the lung and the bioluminescent signals were significantly decreased after 20 days of cofilin Ser3 peptide treatment. In contrast, only saline treatment resulted in metastasis from the right lung to the left lung (FIG. 3B). The ex vivo bioluminescence images also showed that cofilin Ser3 peptide significantly reduced the signal of metastatic lung tumor to the left lung (FIG. 3C). The cofilin Ser3 peptide treatment group significantly reduced the size and the number of lung metastatic lesions (FIG. 3D). Additionally, IHC staining also revealed a significant decrease in the intensity of endogenous CFL-1 serine-3 phosphorylation in lung tumors after 20 days of cofilin Ser3 peptide treatment (FIG. 3E).

To assess toxicity, examination of the liver, spleen, and kidneys revealed no significant morphological changes after cofilin Ser3 peptide treatment (FIG. 3F). Furthermore, the changes of body weights showed no significant different comparing the sham control groups and cofilin Ser3 peptide-injected group (FIG. 3G). At the end of the experiment, blood samples were collected from each animal, and hematological and biochemical analyses were conducted to assess toxicity. The results showed no abnormalities, with any observable differences among the mice remaining within normal ranges (Table 1 and 2).

TABLE 1
Blood hematology parameters after CFL Ser3 peptide treatment.

| | | | Normal saline (n = 4) | | | CFL Ser3 peptide (n = 4) | | |
|---|---|---|---|---|---|---|---|---|
| Hematology | Abb. | Unit | Mean | ± | SEM | Mean | ± | SEM |
| White blood cell | WBC | k/μL | 5.818 | ± | 2.048 | 9.000 | ± | 1.879 |
| Neutrophil | NEU | k/μL | 1.768 | ± | 0.818 | 2.185 | ± | 0.323 |
| Lymphocyte | LYM | k/μL | 3.788 | ± | 1.248 | 6.505 | ± | 1.516 |
| Monocyte | MONO | k/μL | 0.148 | ± | 0.094 | 0.108 | ± | 0.027 |
| Eosinophil | EOS | k/μL | 0.110 | ± | 0.029 | 0.188 | ± | 0.043 |
| Basophil | BASO | k/μL | 0.005 | ± | 0.003 | 0.015 | ± | 0.005 |
| % Neutrophil | % N | % | 27.650 | ± | 3.084 | 25.450 | ± | 2.094 |
| % Lymphocyte | % L | % | 66.300 | ± | 1.187 | 71.125 | ± | 2.190 |
| % Monocyte | % M | % | 3.800 | ± | 2.836 | 1.150 | ± | 0.087 |
| % Eosinophil | % E | % | 2.175 | ± | 0.626 | 2.125 | ± | 0.284 |
| % Basophil | % B | % | 0.075 | ± | 0.048 | 0.150 | ± | 0.029 |
| Red blood cell | RBC | M/μL | 9.955 | ± | 0.909 | 9.693 | ± | 0.485 |
| Hemoglobin | HGB | g/dL | 15.075 | ± | 1.322 | 14.550 | ± | 0.689 |
| Hematocrit | HCT | % | 46.925 | ± | 4.776 | 45.075 | ± | 2.532 |
| Mean corpuscular | MCV | fL | 47.025 | ± | 0.534 | 46.475 | ± | 0.388 |
| volume | | | | | | | | |
| Mean corpuscular | MCH | pg | 15.150 | ± | 0.132 | 15.025 | ± | 0.085 |
| hemoglobin | | | | | | | | |
| Mean corpuscular | MCHC | g/dL | 32.275 | ± | 0.435 | 32.325 | ± | 0.368 |
| hemoglobin | | | | | | | | |
| concentration | | | | | | | | |
| Red cell | RDW | % | 23.000 | ± | 0.897 | 22.525 | ± | 0.743 |
| distribution width | | | | | | | | |
| Reticulocytes | RET | k/μL | 466.200 | ± | 24.153 | 430.200 | ± | 28.848 |
| % Reticulocytes | % R | % | 4.738 | ± | 0.239 | 4.440 | ± | 0.200 |

*Mice were treated with the CFL Ser3 peptide or normal saline every two days via intravenous injection, and blood samples were collected after they were sacrificed. Injected dose was 10 mmol/L/kg. Data are presented as the mean ± SEM.

TABLE 2
Blood biochemical parameters after CFL Ser3 peptide treatment.

| | | | Normal saline (n = 4) | | | CFL Ser3 peptide (n = 4) | | |
|---|---|---|---|---|---|---|---|---|
| Biochemistry | Abb. | Unit | Mean | ± | SEM | Mean | ± | SEM |
| Total-cholesterol | TCHO | mg/dL | 161.25 | ± | 7.16 | 151.25 | ± | 4.96 |
| Albumin | ALB | g/dL | 3.43 | ± | 0.20 | 3.07 | ± | 0.03 |
| Glucose | GLU | mg/dL | 245.50 | ± | 48.20 | 263.75 | ± | 9.62 |
| Aspartate | AST | U/L | 1112.50 | ± | 343.50 | 983.50 | ± | 134.89 |
| aminotransferase | | | | | | | | |
| Alanine | ALT | U/L | 165.00 | ± | 77.00 | 133.00 | ± | 18.45 |
| aminotransferase | | | | | | | | |
| Total bilirubin | TBIL | mg/dL | 3.15 | ± | 1.32 | 1.25 | ± | 0.21 |
| Total protein | TP | g/dL | 5.11 | ± | 0.18 | 4.83 | ± | 0.06 |
| Creatinine | CRE | mg/dL | 0.13 | ± | 0.01 | 0.20 | ± | 0.02 |
| Blood urea nitrogen | BUN | mg/dL | 23.75 | ± | 1.03 | 27.00 | ± | 2.80 |
| Calcium | Ca | mg/dL | 9.45 | ± | 0.30 | 9.88 | | 0.21 |
| Alkaline | ALP | U/L | 177.50 | ± | 8.49 | 140.50 | ± | 5.06 |
| phosphatase | | | | | | | | |
| Phosphate | IP | mg/dL | 14.71 | ± | 1.94 | 8.42 | ± | 0.40 |

*Mice were treated with the CFL Ser3 peptide or normal saline every two days via intravenous injection, and blood samples were collected after they were sacrificed. Injected dose was 10 mmol/L/kg. Data are presented as the mean ± SEM.

After the treatment approach in lung orthotopic mice model, cofilin Ser3 peptide could prolong the mouse survival rate to the control group (FIG. 3H). The median survival time was longer in CFL Ser3 peptide treated tumor-bearing mice than in normal saline control groups (Table 3). Combining these results, the delivery of synthetic cofilin Ser3 peptide efficiently inhibits lung cancer metastasis without adversely affecting normal organs.

TABLE 3
Kaplan-Meier survival curve in the orthotopic lung cancer model.

| | Saline | CFL peptide |
|---|---|---|
| Sample size (n) | 10 | 10 |
| Median survival (Days) | 33.5 | 58 |

| | |
|---|---|
| Significance | p = 0.0407 |
| 95% Confidence interval | 0.09357 to 0.909 |
| Hazard ratio | 0.3424 |

### Embodiment 4: The CFL Ser3 Peptide Inhibit DNA Repair Initiation Through the Suppression of Endogenous Cofilin Phosphorylation

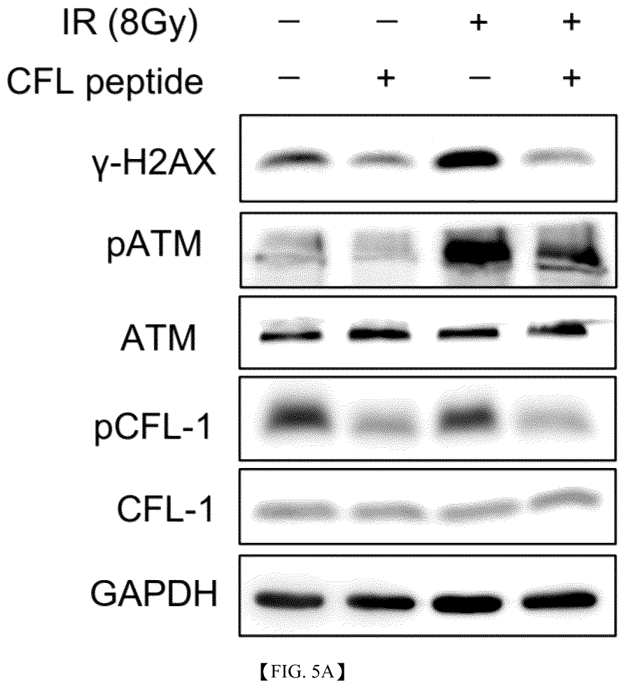

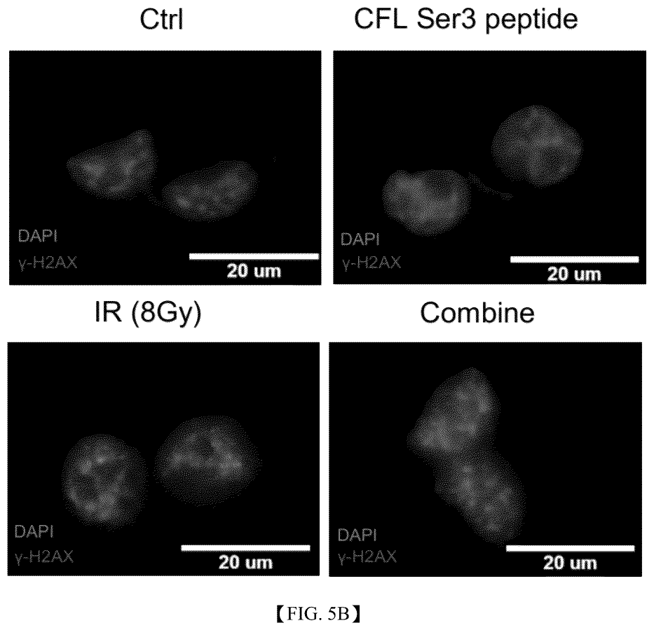

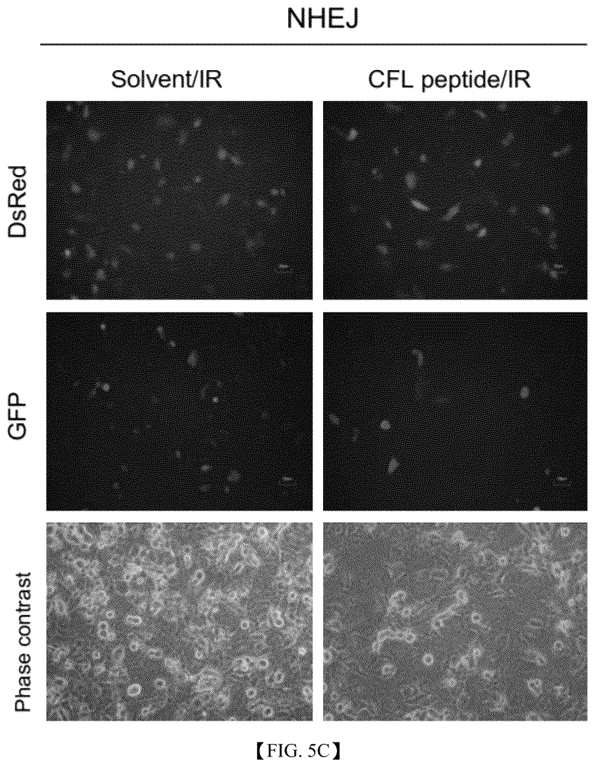

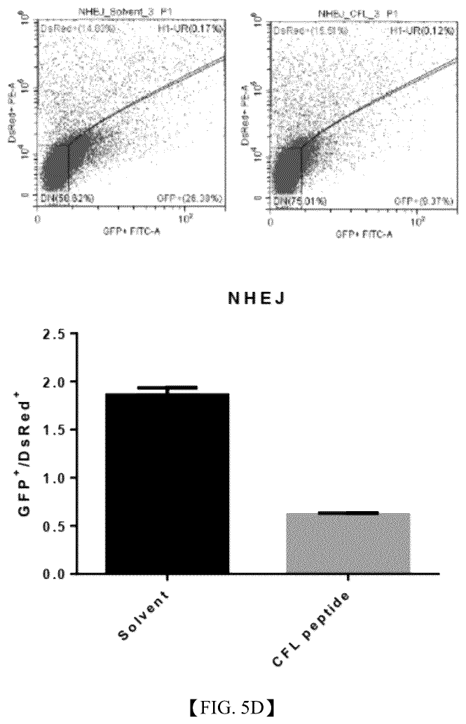

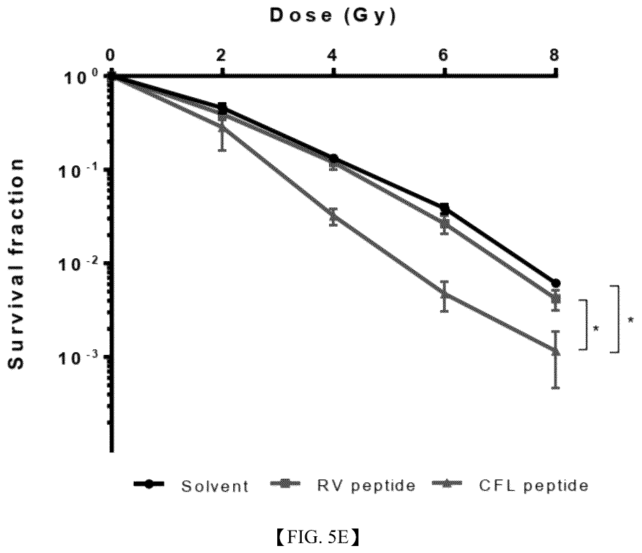

To determine whether the DNA repair capacity is influenced by the phosphorylated form of endogenous CFL-1, the effect of downregulation of endogenous phosphorylated cofilin on the DNA damage response was investigated. Western blot analysis showed that the radiation-induced expression of γ-H2AX was suppressed by CFL Ser3 peptide treatment, but the CFL Ser3 peptide did not influence the activity of ATM kinase (FIG. 5A). The formation of γ-H2AX foci in the nuclei was significantly suppressed by CFL Ser3 peptide treatment after irradiation (FIG. 5B). The DNA repair of I-SceI endonuclease digested GFP expression cassettes was analyzed following CFL Ser3 peptide treatment after exposure to ionizing radiation (FIG. 5C). The results demonstrated that repair capacity of NHEJ (Non-homologous end joining) system was reduced by CFL Ser3 peptide treatment after irradiation (FIG. 5D). Furthermore, the CFL Ser3 peptide which suppressed the endogenous cofilin-1 phosphorylation was able to enhance radiosensitivity in lung cancer cells by reducing the survival fractions compared with solvent control and RV peptide using the colony formation assay (FIG. 5E). These data suggested that the cofilin Ser3 peptide could modulate radiation-induced γ-H2AX formation to influence the radiosensitivity of NSCLC cells by suppressed the endogenous cofilin-1 phosphorylation.

Although the invention has been described with reference to specific embodiments, this description is not meant to be construed in a limiting sense. Various modifications of the disclosed embodiments, as well as alternative embodiments, will be apparent to persons skilled in the art. It is, therefore, contemplated that the appended claims will cover all modifications that fall within the true scope of the invention.

1. A pharmaceutical composition, comprising a peptide with an amino acid sequence consisting of SEQID NO: 1.

2. The pharmaceutical composition of claim 1, wherein the pharmaceutical composition further comprises a cell-membrane penetrating peptide.

3. The pharmaceutical composition of claim 2, wherein the cell-membrane penetrating peptide selected from any one of:
(i) an antennapedia class peptide;
(ii) a protegrin class peptide;
(iii) a HIV-TAT class peptide;
(iv) an amphipathic class peptide selected from an amphipathic and net positively charged peptide and a proline-rich amphipathic peptide;
(v) a peptide exhibiting high α-helical content;
(vi) a peptide comprising oligomers of basic amino acids;
(vii) pVEC;
(viii) a calcitonin-derived peptide; and
(ix) an amphiphilic cyclic cell penetrating sequence.

4. The pharmaceutical composition of claim 3, wherein the cell-membrane penetrating peptide is the antennapedia class peptide.

5. The pharmaceutical composition of claim 4, wherein the cell-membrane penetrating peptide consists of SEQ ID NO: 3.

6. The pharmaceutical composition of claim 1, wherein the peptide is conjugated with a chelating agent selected from the group consisting of DTPA, HEDP, MGDA, DTPMP and combination thereof.

7. The pharmaceutical composition of claim 6, wherein the chelating agent chelates a radionuclide.

8. The pharmaceutical composition of claim 7, wherein the radionuclide is selected from the group consisting of ¹⁷⁷Lu, ^{186/188}Re, ⁶⁷Cu, ^{60/64}Cu, ⁹⁰Y, ⁸⁶Y, ^{44/43/47}Sc, ^{203/212}Pb, ^{67/68}Ga, ^{155/157}Gd, ²¹²Bi, ²²⁵Ac, ²²³Ra, ^{99m}Tc, and ¹¹¹In.

9. The pharmaceutical composition of claim 1, wherein the pharmaceutical composition further comprises a pharmaceutically acceptable excipient, carrier, or diluent.

10. The pharmaceutical composition of claim 1, wherein the pharmaceutical composition further comprises an anti-cancer agent selected from the group consisting of etoposide, doxorubicin, daunorubicin, epirubicin, bleomycin, rituxan, gemcitabine, nivolumab, pembrolizumab, and ipilimumab.

11. A method of treating a cancer disease in a subject, comprising administering to the subject the pharmaceutical composition of claim 1.

12. The method of claim 11, wherein the cancer disease exhibits overexpression of the LIMK gene.

13. The method of claim 12, wherein the cancer disease is selected from the group consisting of colon adenocarcinoma, lymphoid neoplasm diffuse large B-cell lymphoma, esophageal carcinoma, head and neck squamous cell carcinoma, kidney renal papillary cell carcinoma, lung adenocarcinoma, pancreatic adenocarcinoma, rectum adenocarcinoma, skin cutaneous melanoma, stomach adenocarcinoma, thymoma, and uterine carcinosarcoma.

14. The method of claim 11, wherein the subject is human, primate, hamster, rabbit, rodent, bovine, swine, sheep, horse, goat, canine or feline.

15. The method of claim 11, wherein the composition is administered to the subject via any one of intramuscular injection, intravenous injection, and subcutaneous injection.

16. A method of diagnosing a cancer disease in a subject, comprising administering to the subject the pharmaceutical composition of claim 1.

17. The method of claim 16, comprising imaging the subject to detect a signal emitted by the radionuclide.

18. The method of claim 16, wherein the cancer disease exhibits overexpression of the LIMK gene.

19. The method of claim 18, wherein the cancer disease is selected from the group consisting of colon adenocarcinoma, lymphoid neoplasm diffuse large B-cell lymphoma, esophageal carcinoma, head and neck squamous cell carcinoma, kidney renal papillary cell carcinoma, lung adenocarcinoma, pancreatic adenocarcinoma, rectum adenocarcinoma, skin cutaneous melanoma, stomach adenocarcinoma, thymoma, and uterine carcinosarcoma.

20. The method of claim 15, wherein the signal is detected using a technique selected from the group consisting of PET, PET/CT, CT, SPECT, SPECT/CT, and MRI.

21. The method of claim 15, wherein the subject is a human, primate, hamster, rabbit, rodent, bovine, swine, sheep, horse, goat, canine or feline.

22. The method of claim 15, wherein the pharmaceutical composition is administered to the subject via any one of intramuscular injection, intravenous injection, and subcutaneous injection.
